(19) United States
(12) Patent Application Publication
(10) Pub. No.: US 2003/0039850 A1
(43) Pub. Date: Feb. 27, 2003

(54) Modifier for hydrophilicity, hydrophilic modification method and aluminum material or aluminum alloy material modified hydrophilic thereby

(75) Inventors: Toshio Inbe, Yokohama-shi (JP); Susumu Maekawa, Yokohama-shi (JP); Akira Ushio, Hatogaya-shi (JP); Koichi Saito, Tokyo (JP)

Correspondence Address: SUGHRUE MION, PLLC, 2100 Pennsylvania Avenue, NW, Washington, DC, 20037-3213, US

(73) Assignee: NIPPON PAINT CO., LTD.

(21) Appl. No.: 10/105,244

(22) Filed: Mar. 26, 2002

(30) Foreign Application Priority Data
Mar. 27, 2001 (JP) 2001-091049

(51) Int. Cl. B32B 15/04; C22F 1/00; C21D 9/08; C07C 27/10; C07C 29/10

(52) U.S. Cl. 428/457; 568/700

(57) ABSTRACT

A modifier for hydrophilicity and a hydrophilic modification method which are capable of suppressing not only the odor of the resulting hydrophilic coat itself but also the unpleasant odor emanation after exposure to odorants and retaining the hydrophilicity even during a long period of use, and an aluminum material or aluminum alloy material by said hydrophilic modification method, which is used suitably for heat exchangers and the like are provided.

A modifier for hydrophilicity which comprises a hydroxyl group- and ester bond-containing radical polymer having a number average molecular weight of 1,000 to 1,000,000,

said hydroxyl group accounting for 50 to 99.9 mole percent and said ester bond accounting for 0.1 to 50 mole percent respectively, of the sum of the hydroxyl groups and ester bonds in the radical polymer, and

said radical polymer accounting for 0.1 to 30% by mass relative to said modifier for hydrophilicity.

### BACKGROUND OF THE INVENTION

The present invention relates to a modifier for hydrophilicity and a hydrophilic modification method capable of providing good hydrophilicity and odor preventing ability even during a long period of use, and more particularly, to a modifier for hydrophilicity and a hydrophilic modification method used suitably for evaporators and heat exchangers.

### PRIOR ART

Evaporators and heat exchangers generally have a complicated structure, namely they have aluminum fins arranged therein at short intervals for heat exchange and further have aluminum tubes intricately arranged for feeding a cooling medium to these fins. Therefore, the discharge of water condensed upon cooling operation is facilitated by modifying the surface of aluminum fins hydrophilic. However, the above surface of aluminum fins which are modified hydrophilic or the like is exposed to severe conditions, namely to repeated heating and cooling and mixed adhesion of condensed water and dust and microorganisms in the atmosphere. Therefore, it is difficult for that surface to retain hydrophilicity over a long period of time.

Japanese Kokai Publication Hei-05-302042 discloses modifiers for hydrophilicity comprising polyvinyl alcohol, polyvinylpyrrolidone (PVP), nylon, phenolic resin, etc. Japanese Kokai Publication Hei-05-202313, Japanese Kokai Publication Hei-05-214273, JP 2649297 and Japanese Kokai Publication Hei-10-30069 disclose modifiers for hydrophilicity comprising water-dispersible organic resins and silica or alumina. Furthermore, Japanese Kokai Publication Hei-09-087576 discloses compositions for hydrophilic modification comprising minute hydrophilic cross-linked acrylic polymer particles.

Japanese Kokai Publication Hei-07-323500, Japanese Kokai Publication Hei-09-14889 and Japanese Kokai Publication Hei-11-131254 disclose modifiers for hydrophilicity intended for retaining hydrophilicity by crosslinking such polar functional groups as sulfonic acid groups and acrylamide for insolubilization.

Japanese Kokai Publication 2000-262968 discloses a coat for aluminum fins which comprises PVA having a degree of saponification of not less than 90% and a metal salt and are intended for suppressing odor emanation and deterioration in hydrophilicity.

### SUMMARY OF THE INVENTION

The present invention has for its object to provide a modifier for hydrophilicity and a hydrophilic modification method which are capable of suppressing not only the odor of the resulting hydrophilic coat itself but also the unpleasant odor emanation after exposure to odorants and retaining the hydrophilicity even during a long period of use. A further object is to provide an aluminum material or aluminum alloy material by said hydrophilic modification method, which is used suitably for heat exchangers and the like.

The present inventors found that when substrate materials made of aluminum or an aluminum alloy, after treatment for rust prevention, are treated with a modifier for hydrophilicity which comprises a hydroxyl group- and ester bond-containing radical polymer having a number average molecular weight of 1,000 to 1,000,000 which contains the hydroxyl groups accounting for 50 to 99.9 mole percent and the ester bonds accounting for 0.1 to 50 mole percent respectively, of the sum of the hydroxyl groups and ester bonds in the radical polymer, thus coated substrate materials with a hydrophilic coat can suppress possible odor emanation from the substrate materials and from the above hydrophilic coat itself even during a long period of use and, even after exposure to an odorant, such as tobacco, sweat or perfume, can suppress emanation of the unpleasant odor of such odorant and, furthermore, can retain a sufficient hydrophilicity. The present invention has been completed on the basis of such findings.

The present invention thus provides a modifier for hydrophilicity

which comprises a hydroxyl group- and ester bond-containing radical polymer having a number average molecular weight of 1,000 to 1,000,000, said hydroxyl group accounting for 50 to 99.9 mole percent and said ester bond accounting for 0.1 to 50 mole percent respectively, of the sum of the hydroxyl groups and ester bonds in the radical polymer, and

said radical polymer accounting for 0.1 to 30% by mass relative to said modifier for hydrophilicity.

The above radical polymer-is preferably a polyvinyl alcohol with a degree of saponification of not less than 90%.

Preferably, the above modifier for hydrophilicity further comprises at least one member selected from the group consisting of phosphorus compound salts and boron compound salts of Ca, Al, Mg, Fe and Zn in an amount of 0.1 to 200% by mass relative to the above radical polymer based on solid matters.

The above modifier for hydrophilicity further preferably comprises at least one member selected form the group consisting of inorganic crosslinking agents, organic crosslinking agents and coupling agents and may comprise less than 50% by mass, relative to the above radical polymer based on solid matters, of particles having a mean particle diameter of 0.01 to 5 μm.

Preferably, the above modifier for hydrophilicity comprises less than 50% by mass, relative to the above radical polymer based on solid matters, of a hydrophilic organic compound having at least one member selected from the group consisting of hydroxyl, carboxyl, sulfo, amido, amino, phosphonic and nitrile groups and an ether bond. Preferably, the above hydrophilic organic compound has a polyoxyethylene chain.

The invention also provides a hydrophilic modification method

which comprises a step of, after treatment for rust prevention of a substrate material made of aluminum or an aluminum alloy, forming a coat having a solid coat amount of 0.02 to 3 g/m² on the substrate with the modifier for hydrophilicity.

The present invention further provides an evaporator which is treated by the above hydrophilic modification method.

In the following, the present invention is described in detail.

### DETAILED DESCRIPTION OF THE INVENTION

The modifier for hydrophilicity of the invention comprises a hydroxyl group- and ester bond-containing radical polymer. The above-mentioned radical polymer has good hydrophilicity and water resistance, has itself no odor and hardly allows adsorption of odorants thereon, so that the modifier for hydrophilicity of the invention which comprises the above radical polymer is excellent in hydrophilicity and odor preventing ability. The hydrophilic coat obtained therefrom will hardly deteriorate even upon exposure to water drops or to running water and, therefore, the inorganic substances, such as silica, or other residual monomer components which are optionally contained and emanate their own dusty odor and/or the unpleasant odors of substances adsorbed thereon are hardly exposed and, further, the materials treated, such as aluminum, are hardly exposed, so that the treated materials themselves can be prevented from being scattered to emanate dusty odors or being corroded.

The above radical polymer has hydroxyl group and ester bond in side chains. The above radical polymer may have a further functional group, other than the hydroxyl group and ester bond in side chains thereof. The functional group includes hydrophilic functional groups and so on, for example anionic groups such as carboxyl, sulfonic and phosphonic groups; cationic groups such as amino group and imidazole ring; and nonionic groups such as epoxy group and ether bond. The total amount of the hydroxyl groups and ester bonds as side chains in the above radical polymer is preferably 80 to 100 mole percent relative to the whole side chain functional groups, other than hydrocarbon groups such as methyl and ethyl, in the above radical polymer.

In the above radical polymer, the hydroxyl group accounts for 50 to 99.9 mole percent, preferably 60 to 99.9 mole percent, of the sum of the hydroxyl groups and ester bonds in the above radical polymer. When the proportion of hydroxyl groups is less than 50 mole percent, the hydrophilicity of the resulting coat becomes poor. On the other hand, when that exceeds 99.9 mole percent, it becomes difficult, from the production process viewpoint, to produce radical polymers satisfying the above conditions.

The above ester bond accounts for 0.1 to 50 mole percent, preferably 0.1 to 40 mole percent, of the sum of the hydroxyl groups and ester bonds in the above radical polymer. When the proportion of ester bonds is less than 0.1 mole percent, it becomes difficult, from the production process viewpoint, to produce radical polymers satisfying the above conditions. On the other hand, when that exceeds 50 mole percent, the hydrophilicity of resulting coat becomes poor.

The above radical polymer is not particularly restricted but may be any of resins obtainable by radical polymerization. Generally, it is a polymer obtainable by radical polymerization of a monomer or monomers having an unsaturated double bond such as a vinyl group. The side chain functional groups of the above radical polymer may be those contained in the monomer previously and thus directly introduced as side chains by polymerization, or those introduced by a modification after polymer formation.

The monomer component to form the above radial polymer is not particularly restricted but, for example, a vinyl ester is preferred; vinyl acetate or a derivative thereof is more preferred. Furthermore, the monomer component may comprise one or more of acrylic monomers such as (meth)acrylic acid; (meth)acrylate esters such as 2-hydroxyethyl acrylate and 2-hydroxyethyl methacrylate; and acrylonitrile; and so on.

The modification is not particularly restricted but includes, for example, saponification, epoxy modification, silyl modification, thiol modification, carbonyl modification and, further, anionic modifications such as carboxylic acid modification, and cationic modifications such as amine modification.

The hydroxyl groups of the above radical polymer are generally obtainable by saponifying a radical polymer obtainable by using vinyl acetate as a monomer component. They may also be obtainable by radical polymerization of a monomer component comprising a hydroxyl-containing monomer.

From the viewpoint of odor prevention and hydrophilicity providing, it is preferred that the above radical polymer be a polyvinyl alcohol. The above polyvinyl alcohol preferably has a degree of saponification of not less than 90%, more preferably not less than 95%. When the degree of saponification is less than 90%, the hydrophilicity may be poor.

The above polyvinyl alcohol may be used in combination with a partial modification derived from the same which are so modified as to acquire some other function group while having the above-mentioned proportions of hydroxyl groups and ester bonds.

The above-mentioned radical polymer has a number average molecular weight of 1,000 to 1,000,000, preferably 10,000 to 200,000. When it is less than 1,000, the film-forming ability is poor and the hydrophilicity and other physical properties of the coat are also poor. When it exceeds 1,000,000, the resulting modifier for hydrophilicity gives a highly viscous solution, leading to poor workability and poor physical properties of the coat.

The above-mentioned radical polymer accounts for 0.1 to 30% by mass, preferably 0.1 to 20% by mass, relative to the modifier for hydrophilicity of the invention. When this is less than 0.1% by mass, a sufficient level of film-forming ability cannot be obtained and the hydrophilicity and odor preventing ability may possibly to be poor. When it exceeds 30% by mass, aggregation tends to occur in the resulting modifier for hydrophilicity, in some cases leading to inferior workability and poor physical properties of the coat.

Preferably, the modifier for hydrophilicity of the invention further contains a hydrophilic organic compound other than the above-mentioned hydroxyl group- and ester bond-containing radical polymer unless the odor preventing ability is sacrificed. When it contains the above-mentioned other hydrophilic organic compound, the modifier for hydrophilicity of the invention can show further improved hydrophilicity.

The above-mentioned hydrophilic organic compound other than the radical polymer has a hydrophilic functional group, and the above hydrophilic functional group includes, for example, hydroxyl, carboxyl, sulfonic, amide, amino and phosphonic groups, an ether bond, an imidazole ring, and a nitrile group. The above ether bond includes an acetal bond. These hydrophilic functional groups may be used singly or in combination. As the above hydrophilic organic compound, there may be mentioned, for example, polyamides such as water-soluble nylon species; polyacrylic acid, polyvinyl sulfonic acid, polystyrenesulfonic acid, polyvinylimidazole, polyvinylpyrrolidone, polyvinylacetamide, polyamines, polyacrylamide, isoprenoid sulfonic acid polymer, polyvinylformamide, polyallylamine, polyethyleneimine, polyamidines, polyglutamic acid, hydoxyethylcellulose, hydroxypropylmethylcellulose, carboxymethylcellulose, polycarbodiimides, water-soluble polyurethanes, water-soluble polyesters, water-soluble phenolic resins, water-soluble epoxy resins and like resins and modifications thereof; chitosan, alginic acid and like hydrophilic low-molecular compounds.

In the above-mentioned hydrophilic organic compound other than the radical polymer, the above-mentioned ether bond may be a polyoxyalkylene chain. When the modifier contains a polyoxyalkylene chain-containing hydrophilic organic compound, the resulting hydrophilic coat can exhibit more improved hydrophilicity while retaining substantially the same level of odor preventing ability as attainable without addition thereof. The polyoxyalkylene chain-containing hydrophilic organic compound is not particularly restricted but includes, for example, polyethylene oxide, polypropylene oxide, nonionic surfactants, reactive alkylene oxide-based surfactants, ethylene oxide-polypropylene oxide block polymers, ethylene oxide-methylene oxide block polymers, ethylene oxide-derived moiety-containing acrylic resins, ethylene oxide-derived moiety-containing polyester resins, and ethylene oxide-derived moiety-containing polyurethane resins.

When contained in the modifier, the above-mentioned hydrophilic organic compound preferably accounts for less than 50% by mass relative to the resin solid matter in the modifier for hydrophilicity of the invention. When the content exceeds 50% by mass, an odorant adhering to the above hydrophilic organic compound may emanate an unpleasant odor and, at the same time, the incorporated proportion of the above-mentioned radical polymer becomes too low and the modifier as a whole may become inferior in odor preventing ability. A content less than 30% by mass is preferred.

The modifier for hydrophilicity of the present invention preferably contains, in combination with the above-mentioned radical polymer, at least one member selected from the group consisting of phosphorus compound salts and boron compound salts of Ca, Al, Mg, Fe and Zn. When it contains the above phosphorus compound salt and/or boron compound salt, which does not emanate any odor by itself, the modifier for hydrophilicity of the invention shows an improved odor preventing ability, retains the hydrophilicity, and shows an improved film-forming ability.

The phosphorus compound salt mentioned above is not particularly restricted but preferably is a salt of an acid constituted of a phosphorus atom(s) and oxygen atoms, such as an oxo acid of phosphorus, including, for example, phosphoric acid salts, phosphorous (phosphonic) acid salts, hypophosphorous (phosphinic) acid salts, phytic acid salts, and condensed phosphoric acid salts. The above condensed phosphoric acid salts are not particularly restricted but include, for example, metaphosphoric acid salts; and salts of polyphosphoric acids such as pyrophosphoric acid and tripolyphosphoric acid. The above-mentioned phosphorus compound salt is preferably one hardly soluble in water, more preferably one having solubility in water (20° C.) of not more than 1.0 g/100 cm³.

The above-mentioned boron compound salt is not particularly restricted but includes, for example, boric acid salts and borofluorate salts.

The above phosphorus compound salt and boron compound salt preferably have a mean particle diameter of 0.01 to 5 The above phosphorus compound salt and/or boron compound salt preferably accounts for 0.1 to 200% by mass, preferably 5% to 50% by mass, relative to the resin solid matter in the modifier for hydrophilicity of the invention. When it is less than 0.1% by mass, the odor preventing ability and film-forming ability may not be enhanced by the addition of the above phosphorus compound salt and/or boron compound salt. When it exceeds 200% by mass, the phosphorus compound salt and/or boron compound salt itself may emanate an odor or physical properties of the coat may possibly deteriorate.

Preferably, the modifier for hydrophilicity of the invention further comprises at least one member selected from the group consisting of inorganic crosslinking agents, organic crosslinking agents and coupling agents. When it contains the above crosslinking agent and/or coupling agent, the above modifier for hydrophilicity can give hydrophilic coats resistant to deterioration, for example erosion, even when they are exposed to water drops or running water; thus, the hydrophilicity and/or film-forming ability can be further improved.

The above inorganic crosslinking agent is not particularly restricted. Preferred are, however, metal compounds capable of forming complexes with the above-mentioned radical polymer, for example metal compounds containing zirconium, titanium, chromium, aluminum or zinc. Silica may also be used as the above inorganic crosslinking agent. The organic crosslinking agent or coupling agent mentioned above is not particularly restricted but includes those having a functional group capable of reacting with the hydroxyl group or with the modified functional group, for example blocked isocyanates, phenolic resins, melamine, silane coupling agents, titanium coupling agents, epoxy compounds, carbodiimides, thiol compounds, silane compounds, amino resins, carboxylic acids and anhydrides thereof, amines, aziridine compounds and like organic compounds.

The above inorganic crosslinking agent, organic crosslinking agent and/or coupling agent preferably accounts for 0.01 to 20% by mass relative to the resin solid matter in the modifier for hydrophilicity of the invention. When it is less than 0.01% by mass, the hydrophilicity or film-forming ability may not be enhanced by the addition of such crosslinking agent and/or coupling agent. When it exceeds 20% by mass, the odor of such crosslinking agent and/or coupling agent itself may become significant, or may adsorb odorants to emanate an unpleasant odor, or may impair the hydrophilicity.

The modifier for hydrophilicity of the invention may further contain particles having a mean particle diameter of 0.01 to 5 μm. In the present specification, the above-mentioned particles having a mean particle diameter of 0.01 to 5 μm are other than the above-mentioned hydrophilic organic compound, phosphorus compound salts, boron compound salts, crosslinking agents and coupling agents. When it contains the above particles, the modifier for hydrophilicity of the invention can make uneven the surface of the coat obtained upon treatment with the above modifier for hydrophilicity and can improve the hydrophilicity through this unevenness.

The above particles may be of an organic compound or an inorganic compound. As the organic compound, there may be mentioned, for example, acrylic, silicone, melamine and cellulosic polymers in minute particle form; titanium oxide, aluminum oxide, zirconium oxide, glass beads and silica may be mentioned as example of the inorganic compound.

When a mean particle diameter of the above particle is smaller than 0.01 μm, it is difficult to obtain an uneven profile sufficient to improve the hydrophilicity of the hydrophilic coat surface. When it exceeds 5 μm, the unevenness of the above coat surface becomes excessive, so that retention of condensate water may occur on the coat surface or the coat may become poor in appearance in some cases. A mean particle diameter of 0.1 to 2 μm is preferred.

The above particles preferably account for less than 50% by mass relative to the resin solid matter in the modifier for hydrophilicity of the invention. When it is not less than 50% by mass, the above particles themselves may emanate an odor and/or an odorant(s) adsorbed thereon may emanate an unpleasant odor. Preferably, it is lower than 30% by mass.

The modifier for hydrophilicity of the invention may further contain a dispersant so that the above-mentioned radical polymer may be dispersed uniformly. The above dispersant is not particularly restricted but includes, for example, high-molecular polycarboxylic acid alkylolamine salts, acrylic copolymers, polycarboxylic acid amide solutions, aliphatic polybasic carboxylic acids, and high-molecular acid polyester salts. Various surfactants may also be added for attaining dispersion.

Any of various antimicrobial agents may be added to the modifier for hydrophilicity of the invention.

Usable as the above antimicrobial agents are, for example, zinc pyrithione, 2-(4-thiazolyl)-benzimidazole, 1,2-benzisothiazoline, 2-n-octyl-4-isothiazolin-3-one, N-(fluorodichloromethylthio)phthalimide, N,N-dimethyl-N′-phenyl-N′-(fluorodichloromethylthio)sulfamide, methyl 2-benzimidazolecarbamate, bis(dimethylthiocarbamoyl) disulfide, N-(trichloromethylthio)-4-cyclohexane-1,2-dicarboximide, barium metaborate, allyl isothiocyanate; polyoxyalkylenetrialkylammonium salts, organosilicon quaternary ammonium salts, hexamethylenebiguanide hydrochloride, and like quaternary ammonium salts; tri-n-butylteradecylphosphonium chloride and like quaternary phosphonium salts; polyphenolic antimicrobial agents, phenylamide antimicrobial agents, and biguanide antimicrobial agents.

Various additives other than those mentioned above may be incorporated in the modifier for hydrophilicity of the invention according to need. As the above additives, there may be mentioned lubricants, antimicrobial agents, antifungal agents, preservatives, antibacterial agents, surfactants, pigments, dyes, and inhibitors for providing corrosion resistance, etc.

The modifier for hydrophilicity of the invention can be prepared in the conventional manner. For example, there can be used the method which comprises effecting dissolution or dispersion of the above-mentioned radical polymer, if desired together with the other hydrophilic organic compound, in the whole modifier, adding the optional component(s), such as the phosphorus compound salt and/or boron compound salt, particles having a mean particle diameter of 0.01 to 5 μm, crosslinking agent, coupling agent, and other additives, and, if necessary, performing forced dispersion using an ultrasonic dispersion mixer, a dispersion mixer using a micro medium or the like.

The present invention provides a hydrophilic modification method which comprises a step of, after treatment for rust prevention of a substrate material made of aluminum or an aluminum alloy, forming a coat having a solid coat amount of 0.02 to 3 g/m² on the substrate with the modifier for hydrophilicity.

Generally, the above substrate material is subjected in advance to degreasing treatment by cleaning with an acidic or alkaline cleaning agent.

The above acidic cleaning agent is not particularly restricted but includes, for example, acidic aqueous solutions such as those of nitric acid, sulfuric acid and hydrofluoric acid or a combination of these. The acid concentration of the above acidic cleaning agent is preferably 1 to 10 N, more preferably 3 to 6 N. It is also preferable to supplement the above acidic cleaning agent with a salt or acid salt of a metal selected from among iron, nickel, cobalt, molybdenum and cerium.

The above alkaline cleaning agent is not particularly restricted but includes, for example, alkaline aqueous solutions containing at least one of sodium hydroxide, sodium silicate and sodium phosphate. A surfactant may be added to the above alkaline aqueous solutions to thereby increase the detergency thereof.

The method of cleaning the above substrate material is not particularly restricted but may comprise, for example, spraying the substrate material with the above cleaning agent or immersing the substrate material in a bath containing the above cleaning agent. The liquid temperature of the above cleaning agent is preferably 10 to 70° C., and the time of contact with the above cleaning agent is preferably 1 second to 5 minutes. When the liquid temperature is below 10° C. or the above time of contact is shorter than 1 second, the etching may be insufficient and, a temperature exceeding 70° C. or a time exceeding 5 minutes may be unfavorable from the economical viewpoint. The substrate material cleaned in the above manner is washed with water and then subjected to treatment for rust prevention.

The above treatment for rust prevention is carried out in the manner of chemical conversion treatment using a chemical conversion agent. Usable as the above chemical conversion agent are the conventional chromatic chromating agent, phosphoric chromatic agent and nonchromic treatment agent.

The above chromic chromating agent is an aqueous solution containing chromic acid, a fluoride and a strong acid, and includes a reactive chromate and electrolytic chromate whose main component is trivalent chromium, and a coating type chromate in which hexavalent and trivalent chromium are admixed. The above phosphoric chromating agent is a mixed aqueous solution containing chromic acid, orthophosphoric acid and a fluoride. In carrying out chemical conversion treatment with the above chromating agent, it is necessary to control the amounts of the hexavalent chromium ion, phosphate ion and fluoride ion, respectively.

The above nonchromic treatment agent includes a reactive treatment agent and a coating type treatment agent. The above reactive treatment agent includes zirconium salts, titanium salts, silicon salts, boron salts and permanganate salts. Also preferred are fluorides of these salts. The above coating type treatment agent includes, for example, coating type nonchromic treatment agents in which a zirconium salt is admixed with a water-soluble resin. Preferably, at least one member selected from the group consisting of manganese, vanadium, tungsten, molybdenum, titanium and aluminum and compounds containing these atoms, such as manganic acid, permanganic acid, vanadic acid, tungstic acid and molybdic acid, silica, phosphoric acid and phosphate salts is added to the above nonchromic treatment agent where necessary.

The method of chemical conversion treatment of the above substrate material is not particularly restricted but includes, for example, the dipping method and spraying method. In cases where the above substrate material has a complicated profile, such a car evaporator, the dipping method is preferred. In the above chemical conversion treatment, the treatment temperature is preferably at ordinary temperature or a slightly elevated temperature, more preferably at 10 to 70° C., and the treatment time is preferably 1 second to 5 minutes.

As the above-mentioned treatment for rust prevention, a resin primer of the epoxy, urethane or acrylic type may be applied on the substrate.

The chemical conversion coat obtained by the above chemical conversion treatment preferably has a solid coat amount of 10 to 300 mg/m², based on such elements as Cr, Zr or Ti adhered. When the amount is less than 10 mg/m², the rust prevention property may be insufficient. When the amount exceeds 300 mg/m², it is economically disadvantageous and, in addition, may lead to a decrease in hydrophilicity as a result of reaction with the hydrophilic coat. After the above chemical conversion treatment, the substrate is washed with water, if necessary, and then subjected to hydrophilic modification using the above modifier for hydrophilicity.

The above hydrophilic modification method is not particularly restricted but includes, for example, the dipping method and coating method. In cases where the substrate material has a complicated profile, such a car evaporator, the dipping method is preferred. In cases where the substrate material is a fin material, the coating method is preferred. When the dipping method is employed in the above hydrophilic modification, the treatment liquid temperature is preferably about 10 to 60° C., and the treatment time is preferably about 3 seconds to about 5 minutes. A coat having a solid coat amount of 0.02 to 3 g/m² is formed by the above hydrophilic modification. When it is less than 0.02 g/m², the hydrophilic properties will not be produced. When it exceeds 3 g/m², the productivity will decrease. It is preferably 0.05 to 3 g/m², more preferably 0.1 to 1 g/m².

After the above hydrophilic modification, baking is carried out, in the cases where the above substrate material is a molding matter at 100 to 220° C. for 10 to 60 minutes, and in the cases where the above substrate material is a fin material at 100 to 330° C. for 3 to 40 minutes, whereby a hydrophilic coat can be obtained, respectively. At a baking temperature below the above-mentioned infimum, unsatisfactory film-forming ability may result and, at above maximum, the durability of hydrophilicity will decrease. The baking temperature is preferably 120 to 200° C. in the cases where the above substrate material is a molding matter and preferably 180 to 280° C. in the cases where the above substrate material is a fin material.

The modifier for hydrophilicity of the invention comprises a radical polymer which contains hydroxyl group accounting for 50 to 99.9 mole percent and ester bond accounting for 0.1 to 50 mole percent respectively, of the sum of the hydroxyl groups and ester bonds in the molecule, and as a consequence, the hydrophilic coat obtained from the above modifier for hydrophilicity shows very good hydrophilicity and, in addition, as the intrinsic properties of the coat, not only emanation of a dusty or like unpleasant odor but also emanation of the unpleasant odor of the odorant after exposure thereto, are attenuated. The mechanisms by which the hydrophilic modification method of the present invention produces such advantageous effects are not fully clear but may be as follows.

Thus, the above-mentioned radical polymer shows a high level of hydrophilicity owing to the hydroxyl groups contained therein, whereas the above-mentioned radical polymer, specifically polyvinyl alcohol is high in crystallinity and high in density, so that even if an odorant is adhered to the hydroxyl groups, it is presumably not accumulated but is released, hence the unpleasant odor of the above odorant will not be emanated.

Therefore, the modifier for hydrophilicity of the present invention and the hydrophilic modification method using the above modifier for hydrophilicity are suitably used for aluminum or aluminum alloy materials, and more suitably used for, in particular, evaporators such as car evaporators, heat exchangers, car fins, room air conditioners and the like.

The hydrophilic modification method of the present invention which has the constitution mentioned above, will not emanate the dusty odor which is intrinsic in some component(s) in the modifier for hydrophilicity or the unpleasant odor of an odorant(s) adsorbed thereon. Thus, even during a long period of use, the odor of the hydrophilic coat itself as obtained can be suppressed and unpleasant odor emanation after exposure to odorants can be suppressed, while the hydrophilicity can be retained. Therefore, the modifier for hydrophilicity, the hydrophilic modification method and aluminum or aluminum alloy materials treated by the above hydrophilic modification method are suitably used for evaporators, heat exchangers, car fins, room air conditioners and so forth.

### EXAMPLES

The following Examples and Comparative Examples further illustrate the present invention. These Examples are, however, by no means limitative of the scope of the present invention. In each table, each concentration data indicates the concentration (in % by mass) in the modifier for hydrophilicity.

### Examples 1 to 18

(Preparation of modifiers for hydrophilicity)

According to the formulations shown in Table 1, modifiers for hydrophilicity having the respective concentrations (% by mass) given in Table 1 were prepared by blending a radical polymer, a phosphorous compound salt or a boron compound salt, hydrophilic organic compound, a crosslinking agent and so forth. Used as the radical polymer was a polyvinyl alcohol powder [degree of saponification 99%; number average molecular weight 50,000], a carboxylic acid-modified polyvinyl alcohol powder[with 5 moles relative to 100 moles of the sum of the hydroxyl and acetoxy groups in the molecule being modified with acrylic acid; the degree of saponification being 99%; the number average molecular weight being 50,000], or a carbonyl-modified polyvinyl alcohol powder[with 1 moles relative to 100 moles of the sum of the hydroxyl and acetoxy groups in the molecule being modified with carbonyl group; the degree of saponification being 99%; the number average molecular weight being 50,000]. Furthermore, a dispersant [polycarboxylic acid amide solution], a crosslinking phenol [novolak phenol resin, number average molecular weight about 1,000], a polyisocyanate [self-emulsifiable aqueous blocked isocyanate], an acrylic resin [copolymer of methoxypolyoxyethylene methacrylate and acrylic acid (8:2)], polyethylene oxide [PEO, number average molecular weight 100,000], polyacrylamide [homopolymer, number average molecular weight 20,000], polyvinyl sulfonic acid [homopolymer, number average molecular weight 20,000], resin particles [methyl methacrylate resin particles, mean particle diameter 1 μm], an antimicrobial agent [2-(4-thiazolyl)benzimidazole] and so forth were used.

(Hydrophilic treatment)

An aluminum alloy-made car evaporator was immersed in a bath containing an acidic cleaning solution containing 10% by mass (1.6 N) of nitric acid and 5% by mass (1.0 N) of sulfuric acid and warmed to 65° C. for 4 minutes, then drawn up, and thoroughly washed with tap water. Further, this car evaporator was immersed in a bath containing a zirconium-containing rust preventing agent (Alsurf 440N, 2%, product of Nippon Paint) warmed to 50° C. for 90 seconds for chemical conversion treatment, and then thoroughly washed with tap water. Then, this car evaporator was immersed in a bath containing one of the modifiers for hydrophilicity obtained in the above manner at 20° C. for 1 minute, then drawn up, and heated to 140° C. (temperature arrived at) for 30 minutes to give a hydrophilic modification product with a solid coat amount of 0.3 g/m².

(Evaluation)

The hydrophilic modification products obtained in the above manner were evaluated for hydrophilicity and odor by the following methods. The results are shown in Table 1.

1. Hydrophilicity evaluation (1)

Hydrophilic modification products were brought into contact with running tap water for 72 hours and then measured for contact angle with water drops. A smaller contact angle can be said to be indicative of a higher level of hydrophilicity.

2. Hydrophilicity evaluation (2)

Test pieces modified for hydrophilicity as mentioned above were brought into contact with running water for 8 hours, then placed in a mixture of 5 grams each of dioctyl phthalate, hexadecanol and stearic acid placed as contaminants in a dish, and the test pieces in the dish were dried at 80° C. for 16 hours (some of the contaminants evaporated). This procedure of contacting with running water and drying was taken as one cycle. After 5 cycles, each test piece was measured for contact angle to thereby evaluate the deterioration in hydrophilicity as a result of contamination in the atmospheric environment.

3. Dusty odor evaluation

Hydrophilic modification products were brought into contact with running tap water for 72 hours and then smelled for evaluation on the following scoring scale:

| | |
|---|---|
| 0 point | No odor; |
| 1 point | An odor is smelled just faintly; |
| 2 points | An odor is smelled rather readily; |
| 3 points | An odor is smelled clearly; |
| 4 points | A strong odor is smelled; |
| 5 points | A very strong odor is smelled. |

4. Adhering odor (1)

The hydrophilic modification product was brought into contact with running tap water for 72 hours and then placed in a meeting room (50 m³) where the product was exposed to the smoke from 20 cigarettes for 3 hours. Then, it was allowed to stand in another smoke-free meeting room (50 m³) for 1 hour and, thereafter, subjected to sensory evaluation on the scale of 0 to 5 as mentioned above under 3.

5. Adhering odor (2)

The hydrophilic modification product was brought into contact with running tap water for 72 hours and then placed in a meeting room (50 m³) where the product was exposed to the odor from 100 ml each of four commercially available perfumes, namely CKone (Calvin Klein), Ptisenbon (Givenchy), True Love (Elizabeth Arden) and Tendre Poison (Christian Dior) . Then, it was allowed to stand in another odor-free meeting room (50 m³) for 1 hour, and subjected to sensory evaluation on the scale of 0 to 5 as mentioned above under

TABLE 1

| | Modifier for hydrophilicity | | | | | | Odor | | |
|---|---|---|---|---|---|---|---|---|---|
| | (concentration in a modifier for hydrophilicity; % by mass) | | | | Hydrophilicity | | Dusty | Adhering | Adhering |
| | PVA | Metal compound | Crosslinking agent | Additive | 1 | 2 | odor 1 | odor 1 | odor 2 |
| Ex.1 | (2.0) | Ca phosphate(0.5) | — | — | 30 | 38 | 1.5 | 1.8 | 1.8 |
| Ex.2 | (2.0) | Fe phosphate(0.5) | — | — | 30 | 38 | 1.5 | 1.8 | 2 |
| Ex.3 | (2.0) | Al tripolyphosphate | — | — | 30 | 38 | 1.5 | 1.8 | 1.8 |
| Ex.4 | (2.0) | Mg borate | — | — | 30 | 37 | 1.5 | 1.8 | 1.8 |
| Ex.5 | (2.0) | Zinc pyrophosphate | — | — | 30 | 38 | 1.5 | 1.8 | 1.8 |
| Ex.6 | (2.0) | Ca phosphate(0.5) | — | — | 28 | 35 | 1.5 | 1.8 | 1.8 |
| | | Dispersant(0.2) | | | | | | | |
| Ex.7 | (2.0) | Ca phosphate(0.5) | Crosslinking | — | 30 | 38 | 1 | 1.8 | 1.8 |
| | | | phenol(0.2) | | | | | | |
| Ex.8 | Carboxylic acid- | Ca phosphate(0.5) | Silane coupling | — | 25 | 35 | 1.5 | 2 | 2 |
| | modified (2.0) | | agent (0.2) | | | | | | |
| Ex.9 | Carbonyl- | Ca phosphate(0.5) | Carbodihydrazide | — | 28 | 35 | 1.5 | 1.8 | 2 |
| | modified (2.0) | | (0.2) | | | | | | |
| Ex.10 | (2.0) | — | Zircon antimony | — | 30 | 38 | 1.5 | 2 | 2 |
| | | | fluoride (0.2) | | | | | | |
| Ex.11 | (2.0) | Ca phosphate(0.5) | Polyisocyanate | — | 30 | 38 | 1 | 1.8 | 1.8 |
| | | | (0.2) | | | | | | |
| Ex.12 | (2.0) | Ca phosphate(0.5) | — | Acrylic resin (0.5) | 25 | 35 | 1 | 1.8 | 1.8 |
| Ex.13 | (2.0) | — | — | Acrylic resin (0.5) | 25 | 35 | 1 | 2 | 2 |
| Ex.14 | (2.0) | Ca phosphate(0.5) | — | PEO (0.5) | 28 | 38 | 1.5 | 1.8 | 2 |
| Ex.15 | (2.0) | Ca phosphate(0.5) | — | Polyacrylamide (0.5) | 25 | 35 | 1.5 | 2 | 2 |
| Ex.16 | (2.0) | Ca phosphate(0.5) | — | Na polyvinylsulfonate | 25 | 35 | 1.5 | 2 | 2 |
| | | | | (0.5) | | | | | |
| Ex.17 | (2.0) | Ca phosphate(0.5) | — | Resin particles (0.5) | 28 | 35 | 1.5 | 1.8 | 2 |
| Ex.18 | (2.0) | Ca phosphate(0.5) | — | Antimicrobial agent | 30 | 38 | 1.5 | 1.8 | 1.8 |
| | | | | (0.2) | | | | | |

### Comparative Examples 1 to 4

Hydrophilic modification products were obtained and evaluated in the same manner as in Example 1 except that modifiers for hydrophilicity respectively having the compositions shown in Table 2 are used. Used in the modifiers for hydrophilicity were polyacrylic acid (homopolymer, number average molecular weight 100,000), colloidal silica [aqueous dispersion of SiO₂, mean particle diameter 0.3 μm], carboxymethylcellulose [number average molecular weight 20,000], polyvinylsulfonic acid [homopolymer, number average molecular weight 20,000], polyacrylamide [homopolymer, number average molecular weight 20,000], polyvinylpyrrolidone [homopolymer, number average molecular weight 20,000] and polyethylene oxide [PEO, number average molecular weight 100,000]. The evaluation results are shown in Table 2.

TABLE 2

| | | Hydro- | | Odor | | |
|---|---|---|---|---|---|---|
| | Modifier for hydrophilicity | philicity | | Dusty | Adhering | Adhering |
| | (concentration in a modifier for hydrophilicity; % by mass) | 1 | 2 | odor 1 | odor 1 | odor 2 |
| Compar. | Polyacrylic acid (1.0) Colloidal silica (2.0) | 20 | 35 | 3 | 3 | 3 |
| Ex.1 | | | | | | |
| Compar. | Carboxymethylcellulose (1.0) Polyacrylic acid (1.0) Zircon antimony fluoride (0.2) | 25 | 50 | 2 | 3 | 2.5 |
| Ex.2 | | | | | | |
| Compar. | Polyvinylsulfonic acid (1.0) Polyacrylamide (1.0) Zircon antimony fluoride (0.2) | 20 | 35 | 1.5 | 3 | 2.5 |
| Ex.3 | | | | | | |
| Compar. | Polyvinylpyrrolidone (1.0) PEO (1.0) Polyacrylic acid (1.0) Ca phosphate (0.2) | 25 | 40 | 1.5 | 2.5 | 3 |
| Ex.4 | | | | | | |

From Table 1 and Table 2, it was clear that while the hydrophilic modification products obtained in Comparative Examples which were outside the scope of the present invention were poor in hydrophilicity and/or odor preventing ability, the hydrophilic modification products obtained in Examples within the scope of the present invention were superior in hydrophilicity and odor preventing ability.

### Examples 19 to 31

Substrates were treated with an alkaline cleaning agent [Surfcleaner 340, 1%, product of Nippon Paint] at 65° C. for 5 seconds and then subjected to chemical conversion treatment with a chromium phosphate-based rust preventing agent [Alsurf 407/47, 3%/0.5%, products of Nippon Paint] at 50° C. for 5 seconds. Then, the modifiers for hydrophilicity having the respective compositions shown in Table 3 in which the described components were among the components used in the modifiers for hydrophilicity of Examples 1 to 18 were applied to the substrates using a bar coater. The coats were then baked at 220° C. for 20 seconds to give hydrophilic modified products, the solid coat amount being adjusted to 0.3 g/m². The hydrophilic treatment products obtained were evaluated in the same manner as in Example 1. The evaluation results are shown in Table 3.

TABLE 3

| | Modifier for hydrophilicity | | | | | | Odor | | |
|---|---|---|---|---|---|---|---|---|---|
| | (concentration in a modifier for hydrophilicity; % by mass) | | | | Hydrophilicity | | Dusty | Adhering | Adhering |
| | PVA | Metal compound | Crosslinking agent | Additive | 1 | 2 | odor 1 | odor 1 | odor 2 |
| Ex.19 | (2.0) | Ca phosphate(0.5) | — | — | 25 | 35 | 1.5 | 1.8 | 1.8 |
| Ex.20 | (2.0) | Carbodihydrazide | — | — | 25 | 35 | 1.5 | 1.8 | 1.8 |
| Ex.21 | (2.0) | Zinc pyrophosphate | — | — | 28 | 35 | 1.5 | 1.8 | 1.8 |
| Ex.22 | (2.0) | Ca phosphate(0.5) | — | — | 25 | 35 | 1.5 | 1.8 | 1.8 |
| | | Dispersant(0.2) | | | | | | | |
| Ex.23 | (2.0) | Ca phosphate(0.5) | Crosslinking | — | 28 | 38 | 1 | 1.8 | 1.8 |
| | | | phenol(0.2) | | | | | | |
| Ex.24 | Carbonyl- | Ca phosphate(0.5) | Carbodihydrazide | — | 22 | 35 | 1.5 | 2 | 1.8 |
| | modified (2.0) | | (0.2) | | | | | | |
| Ex.25 | (2.0) | — | Zircon antimony | — | 25 | 35 | 1.5 | 2 | 2 |
| | | | fluoride (0.2) | | | | | | |
| Ex.26 | (2.0) | Ca phosphate(0.5) | — | Acrylic resin (0.5) | 20 | 33 | 1 | 1.8 | 1.8 |
| Ex.27 | (2.0) | — | — | Acrylic resin (0.5) | 23 | 33 | 1 | 2 | 2 |
| Ex.28 | (2.0) | Ca phosphate(0.5) | — | PEO (0.5) | 23 | 35 | 1.5 | 2 | 1.8 |
| Ex.29 | (2.0) | Ca phosphate(0.5) | — | Polyacrylamide (0.5) | 20 | 33 | 1.5 | 2 | 2 |
| Ex.30 | (2.0) | Ca phosphate(0.5) | — | Na polyvinylsulfonate | 20 | 33 | 1.5 | 2 | 2 |
| | | | | (0.5) | | | | | |
| Ex.31 | (2.0) | Ca phosphate(0.5) | — | Resin particles (0.5) | 20 | 30 | 1.5 | 2 | 1.8 |

From Table 1 and Table 3, it is clear that, in Examples 19 to 31, where degreasing treatment, treatment for rust prevention and baking conditions were changed, the hydrophilicity was improved in most cases as compared with Examples 1 to 18, although the odor preventing ability remained almost the same.

1. A modifier for hydrophilicity
which comprises a hydroxyl group- and ester bond-containing radical polymer having a number average molecular weight of 1,000 to 1,000,000,
said hydroxyl group accounting for 50 to 99.9 mole percent and said ester bond accounting for 0.1 to 50 mole percent respectively, of the sum of the hydroxyl groups and ester bonds in the radical polymer, and
said radical polymer accounting for 0.1 to 30% by mass relative to said modifier for hydrophilicity.

2. The modifier for hydrophilicity according to claim 1,
wherein the radical polymer is a polyvinyl alcohol with a degree of saponification of not less than 90%.

3. The modifier for hydrophilicity according to claim 1 or 2,
which comprises at least one member selected from the group consisting of phosphorus compound salts and boron compound salts of Ca, Al, Mg, Fe and Zn in an amount of 0.1 to 200% by mass relative to a resin solid matter.

4. The modifier for hydrophilicity according to any of claims 1 to 3,
which comprises at least one member selected from the group consisting of inorganic crosslinking agents, organic crosslinking agents and coupling agents.

5. The modifier for hydrophilicity according to any of claims 1 to 4,
which comprises less than 50% by mass, relative to the resin solid matter, of particles having a mean particle diameter of 0.01 to 5 μm.

6. The modifier for hydrophilicity according to any of claims 1 to 5,
which comprises less than 50% by mass, relative to the resin solid matter, of a hydrophilic organic compound having at least one member selected from the group consisting of hydroxyl, carboxyl, sulfo, amido, amino, phosphonic and nitrile groups and an ether bond.

7. The modifier for hydrophilicity according to claim 6,
wherein the hydrophilic organic compound has a polyoxyethylene chain.

8. A hydrophilic modification method
which comprises a step of, after treatment for rust prevention of a substrate material made of aluminum or an aluminum alloy, forming a coat having a solid coat amount of 0.02 to 3 g/m² on the substrate with the modifier for hydrophilicity according to any of claims 1 to 7.

9. An aluminum or aluminum alloy material treated by the hydrophilic modification method according to claim 8.
